(19) United States
(12) Patent Application Publication
(10) Pub. No.: US 2023/0363624 A1
(43) Pub. Date: Nov. 16, 2023

(54) ENDOSCOPE

(71) Applicant: OLYMPUS MEDICAL SYSTEMS CORP., Tokyo (JP)

(72) Inventors: Satoru YAJIMA, Tokyo (JP); Kazuki MINAMIMOTO, Ebina-shi (JP); Shinnosuke OHIGASHI, Tokyo (JP)

(73) Assignee: OLYMPUS MEDICAL SYSTEMS CORP., Tokyo (JP)

(21) Appl. No.: 18/144,877

(22) Filed: May 9, 2023

Related U.S. Application Data
(60) Provisional application No. 63/340,521, filed on May 11, 2022.

(51) Int. Cl. A61B 1/00; A61B 17/29; A61M 25/09

(52) U.S. Cl. CPC A61B 1/00098; A61B 17/29; A61M 25/0905

(57) ABSTRACT

An endoscope of the disclosure includes an insertion section including a distal end portion, a bending portion, and a flexible tube portion. The distal end portion includes: a first opening connected to an instrument channel, a second opening connected to a wire channel, a wire, a first detent surface, and a raising base rotatable about an axis between a lowered position and a raised position. The raising base includes an attachment housing and a distal end of the wire is attached to the raising base at the attachment housing. The wire is movable within the wire channel to rotate the raising base about the axis from the lowered position to the raised position, and in the raised position, a stop surface of the attachment housing contacts the first detent surface to limit an amount of rotation of the raising base.

### RELATED APPLICATION DATA

This application is based on and claims priority under 37 U.S.C. §119 to U.S. Provisional Application No. 63/340,521 filed on May 11, 2022, the entire contents of which are incorporated herein by reference.

### FIELD OF DISCLOSURE

The disclosure is related to an endoscope that includes a forceps raising base (forceps elevator) that changes a protruding direction of a treatment instrument or the like protruding outward from a distal end structural portion of an insertion section.

### BACKGROUND

Endoscopes have been used in a medical field, an industrial field, and the like. An endoscope includes an insertion section that is formed to have an elongated tube shape and that is inserted inside a subject, and a distal end structural portion is provided at a distal end of the insertion section. Furthermore, an image pickup unit, an illumination unit and the like are provided in the distal end structural portion, for example.

Moreover, an insertion section of such an endoscope sometimes includes an elongated tubular conduit (a so-called treatment instrument insertion conduit). A treatment instrument and the like may be inserted through the conduit. The treatment instrument and the like here are instruments that include forceps or the like at a distal end and that are used at the time of performing various treatments such as biopsy of sampling a part of living tissue inside a body cavity, resection of a lesion inside the body cavity, and the like.

Moreover, the treatment instrument or the like may be inserted into the conduit in a state where the insertion section is inserted in the body cavity of a subject, and a distal end portion of the treatment instrument or the like may be caused to protrude outward from the distal end structural portion. Various treatments using the treatment instrument and the like may be performed in such a state.

Moreover, there have been disclosed endoscopes that mainly take duodenum or the like as a treatment target, and that include, at the distal end structural portion, a forceps raising base for changing a protruding direction of the treatment instrument or the like protruding outward from the distal end structural portion. Various proposals regarding such an endoscope are made in Japanese Patent Application Laid-Open Publication No. 2015-165839, Japanese Patent Application Laid-Open Publication No. H08-56900, Japanese Patent Application Laid-Open Publication No. H04-218134, Japanese Patent No. 3159464, and the like.

Japanese Patent Application Laid-Open Publication No. 2015-165839, Japanese Patent Application Laid-Open Publication No. H08-56900, and the like each disclose an endoscope where the forceps raising base and a mechanism section for operating the forceps raising base are disposed in respective spaces that are partitioned by a wall member or the like.

Furthermore, Japanese Patent Application Laid-Open Publication No. H04-218134, Japanese Patent No. 3159464, and the like each disclose an endoscope where a partitioning wall is not provided between the forceps raising base and a forceps raising base operation mechanism, where the endoscope includes a mechanism for directly pulling the forceps raising base, the forceps raising base being connected to an operation wire. In relation to such an endoscope, a configuration is disclosed in which a part of a rotation restriction portion for restricting a rotation range of the forceps raising base is disposed in a movable region of the treatment instrument.

### SUMMARY

An endoscope of an aspect of the present disclosure includes an insertion section including a distal end portion, a bending portion, and a flexible tube portion. The bending portion is between the distal end portion and the flexible tube portion. The flexible tube portion is at a proximal end side of the insertion section and the distal end portion at a distal end side of the insertion section. The distal end portion includes: a first opening connected to an instrument channel, a second opening connected to a wire channel, a wire located in the wire channel and protruding distally from the second opening, a first detent surface, and a raising base rotatable about an axis between a lowered position and a raised position. In the raised position, the raising base intersects a projection of an axis of the instrument channel extending from the first opening. The raising base includes an attachment housing and a distal end of the wire is attached to the raising base at the attachment housing. And the wire is movable within the wire channel to rotate the raising base about the axis from the lowered position to the raised position, and, in the raised position, a stop surface of the attachment housing contacts the first detent surface to limit an amount of rotation of the raising base.

### BRIEF DESCRIPTION OF THE DRAWINGS

FIG. 1 is a diagram schematically showing an entire endoscope system including an endoscope of an embodiment of the present disclosure.

FIG. 2 is an enlarged perspective view of main parts of a distal end structural portion of the endoscope of the embodiment of the present disclosure.

FIG. 3 is an enlarged perspective view of main parts of the distal end structural portion of the endoscope of the embodiment of the present disclosure.

FIG. 4 is an exploded perspective view showing a state where a distal end cover of the distal end structural portion in FIG. 2 is removed.

FIG. 5 is a perspective view showing, in an enlarged manner, only a forceps raising base of the distal end structural portion in FIG. 2.

FIG. 6 is an enlarged perspective view showing a configuration of the forceps raising base and a forceps raising base operation mechanism of the distal end structural portion in FIG. 2.

FIG. 7 is an enlarged plan view seen in an arrow [7] direction in FIG. 6.

FIG. 8 is a perspective view showing an internal structure of the distal end structural portion in FIG. 2.

FIG. 9 is an enlarged perspective view of main parts showing a structure for attaching an operation wire to a wire pulling portion of the forceps raising base of the distal end structural portion in FIG. 2.

FIG. 10 is a diagram showing a modification of the structure, in FIG. 9, for attaching the operation wire to the wire pulling portion.

FIG. 11 is a diagram showing a state where the forceps raising base of the distal end structural portion is raised halfway.

FIG. 12 is a diagram showing a state where the forceps raising base of the distal end structural portion from which the distal end cover is removed is raised halfway.

FIG. 13 is a diagram showing a maximum raised state of the forceps raising base of the distal end structural portion.

FIG. 14 is a diagram showing the maximum raised state of the forceps raising base of the distal end structural portion from which the distal end cover is removed.

FIG. 15 is a diagram showing a state where the forceps raising base is lowered together with a treatment instrument at the distal end structural portion from which the distal end cover is removed.

FIG. 16 is a diagram showing a state where the treatment instrument reached a maximum raised position at the distal end structural portion from which the distal end cover is removed.

FIG. 17 is a diagram showing a modification of the distal end structural portion of the endoscope of the embodiment of the present disclosure.

FIG. 18 is a diagram schematically showing an external appearance of the distal end structural portion of the endoscope of the embodiment of the present disclosure.

FIG. 19 is a diagram showing a cross-sectional surface along a line [18]-[18] in FIG. 18.

FIG. 20 is a diagram showing a cross-sectional surface along a surface indicated by [20]-[20] in FIG. 19.

FIG. 21 is a diagram showing a cross-sectional surface along a line [21]-[21] in FIG. 18.

### DETAILED DESCRIPTION

Generally, as a structure of a conventional endoscope, structures disclosed in Japanese Patent Application Laid-Open Publication No. 2015-165839, Japanese Patent Application Laid-Open Publication No. H08-56900, and the like described above may be cited. In the case of such an endoscope where a forceps raising base and a mechanism section for operating the forceps raising base are disposed in respective spaces partitioned by a wall member or the like, a structure of a distal end structural portion including the mechanism section for operating the forceps raising base may possibly become complex.

Accordingly, for a simpler structure, structures of endoscopes disclosed in Japanese Patent Application Laid-Open Publication No. H04-218134, Japanese Patent No. 3159464, and the like may be cited, for example. The distal end structural portion of such an endoscope has a structure where a partitioning wall is not provided between the forceps raising base and a forceps raising base operation mechanism, where the endoscope includes a mechanism for directly pulling the forceps raising base, the forceps raising base being connected to an operation wire.

However, in the case of adopting a structure where a partitioning wall is not provided between the forceps raising base and the forceps raising base operation mechanism, a structure near a distal end opening of the distal end structural portion, the operation wire or the like may possibly obstruct insertion/removal of a treatment instrument or the like. For example, a cause of occurrence of such a phenomenon may be that a rotation restriction portion near the distal end opening of the distal end structural portion, the operation wire and the like exist in a movable region of the treatment instrument or the like.

According to an embodiment of the present disclosure described below, there may be provided an endoscope in which the structure of the distal end structural portion is simplified, and in which insertion/removal of a treatment instrument or the like is not obstructed and operability may be secured in relation to the treatment instrument or the like.

In the following, the present disclosure will be described using an embodiment shown in the drawings. Each diagram used in the description below is schematic, and to allow each structural component to be shown large enough to be recognized in the drawings, a dimensional relationship, scales, and the like of respective members may be shown differently for each structural component. Accordingly, the present disclosure is not limited to the mode shown in the drawings with respect to the number of pieces of each structural component, the shape of each structural component, a ratio of respective sizes of structural components, relative positional relationships of respective structural components, and the like shown in each drawing.

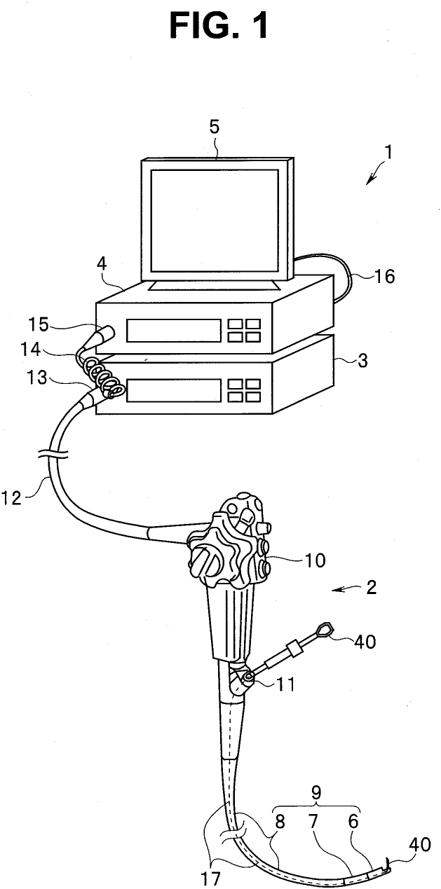

First, a schematic configuration of an endoscope system including an endoscope of an embodiment of the present disclosure will be described below with reference to FIG. 1. FIG. 1 is a diagram schematically showing the entire endoscope system including the endoscope of the embodiment of the present disclosure. Note that a basic configuration of an endoscope system 1 shown in FIG. 1 includes the same configuration as the configuration of a conventional endoscope system. Accordingly, in the following, only a brief description of each structural member of the endoscope system 1 will be given.

As shown in FIG. 1, the endoscope system 1 of the present embodiment includes an endoscope 2, a light source device 3, a video processor 4, a display device 5, a treatment instrument 40 for endoscope, and the like.

The endoscope 2 includes an insertion section 9 having an elongated tube shape, an operation section 10, a universal cord 12, and the like.

The insertion section 9 is a structural member that is inserted into a subject. The insertion section 9 is formed from a distal end structural portion (distal end portion) 6, a bending portion 7, and a flexible tube portion 8 that are continuously provided in a stated order from a distal end side. The bending portion 7 is between the distal end portion 6 and the flexible tube portion 8, the flexible tube portion 8 is at a proximal end side of the insertion section 9 and the distal end portion 6 at a distal end side of the insertion section 9. Accordingly, the insertion section 9 is formed into an elongated tube shape as a whole. The operation section 10 is further connected on a proximal end side of the insertion section 9. Furthermore, although details will be given later, the endoscope 2 allows insertion of the treatment instrument 40 for endoscope (hereinafter simply referred to as “treatment instrument”). To this end, a treatment instrument insertion channel 17 that is a conduit that allows insertion of the treatment instrument 40 is provided in the insertion section 9, from a distal end to a proximal end.

Various structural members such as an image pickup unit, an illumination unit, and a forceps raising base (not shown in FIG. 1; described in detail later) are provided inside the distal end structural portion 6.

The operation section 10 includes an operation section main body, a plurality of operation members, a forceps insertion port 11, and the like. The operation section main body is substantially box-shaped as a whole, and forms a grasping portion. As described above, the insertion section 9 is provided extending from the operation section main body. The plurality of operation members are operation members used to perform various operations of the endoscope 2. The plurality of operation members is provided on an outer surface of the operation section main body.

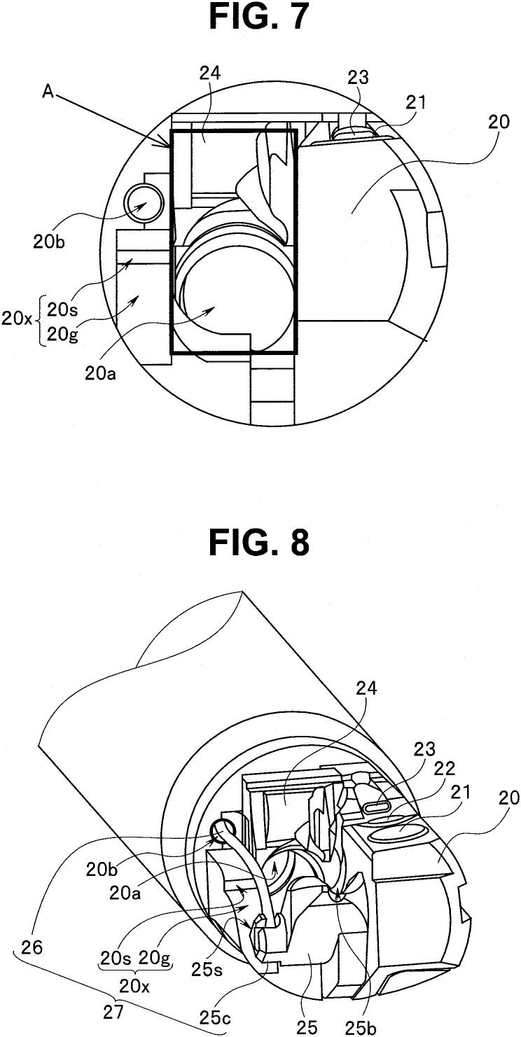

The forceps insertion port 11 is provided on the operation section main body of the operation section 10, close to a distal end. The forceps insertion port 11 is a proximal end side opening of the treatment instrument insertion channel (instrument channel) 17 of the insertion section 9. Furthermore, a distal end side of the treatment instrument insertion channel 17 is connected to a first distal-end-side opening (first opening) 20a (not shown in FIG. 1; described later; see FIGS. 7 and 8) of the distal end structural portion 6. According to such a configuration, a distal end of the treatment instrument 40 inserted from the forceps insertion port 11 protrudes outward from the first distal-end-side opening 20a of the distal end structural portion 6.

The universal cord 12 is a tubular member that extends from a side of the operation section 10. A scope connector 13 is provided at a distal end of the universal cord 12. The scope connector 13 is connected to the light source device 3.

The light source device 3 is a device configured to supply illumination light to the illumination unit provided inside the distal end structural portion 6 of the insertion section 9 of the endoscope 2. The illumination light emitted from the light source device 3 is transmitted from the light source device 3 to the illumination unit in the distal end structural portion 6 of the insertion section 9 of the endoscope 2, through an optical fiber cable or the like (not shown) disposed from the scope connector 13 through the universal cord 12, the operation section 10, and the insertion section 9. The illumination light further passes through an optical illumination member provided in the distal end structural portion 6, and is radiated toward an observation target object near the distal end structural portion 6.

Note that a configuration for transmitting the illumination light from the light source device 3 to the distal end structural portion 6 through the optical fiber cable or the like is cited as an example of the illumination unit, but such a configuration is not restrictive. For example, an LED (light emitting diode) or the like as an illumination light source may be provided inside the distal end structural portion 6, and light emission of the illumination light source (LED) may be controlled by the light source device 3.

A scope cable 14 extends sideways from the scope connector 13. An electrical connector section 15 is provided at a distal end of the scope cable 14. The electrical connector section 15 is connected to the video processor 4.

The video processor 4 is a control device including a control circuit configured to control the entire endoscope system 1, and the like. In this case, the video processor 4 includes a signal processing circuit, a control processing circuit, and the like. The signal processing circuit is configured to receive an image pickup signal from the image pickup unit (not shown) provided inside the distal end structural portion 6 of the insertion section 9 of the endoscope 2, and to perform signal processing, for example. The control processing circuit is configured to output a control signal for driving the image pickup unit, for example.

The video processor 4 and the image pickup unit are electrically connected by a signal transmission cable (not shown). For this purpose, the signal transmission cable is disposed inserted from the electrical connector section 15 to the image pickup unit in the distal end structural portion 6 of the insertion section 9, through the scope cable 14, the scope connector 13, the universal cord 12, and the operation section 10. According to such a configuration, the image pickup signal outputted from the image pickup unit, the control signal outputted from the video processor 4, and the like are transmitted between the image pickup unit and the video processor 4 through the signal transmission cable. Note that as a mode of the signal transmission cable, a complex cable in which a plurality of cables is bundled and covered by an outer shield, an outer tube or the like is adopted, for example.

The video processor 4 and the display device 5 are connected using a video cable 16. The video cable 16 transmits, to the display device 5, an image signal, a control signal and the like outputted from the video processor 4.

The display device 5 receives the image signal and the control signal outputted from the video processor 4, and performs display of an endoscopic image of a mode, various information and the like in a display mode according to the control signal that is received.

Moreover, with the endoscope system 1 of the present embodiment, the treatment instrument 40 is removably inserted into the endoscope 2. FIG. 1 shows a state where the treatment instrument 40 is inserted up to a position in the endoscope 2. As the treatment instrument 40, treatment instruments of various modes are prepared, and a treatment instrument is selected and used as appropriate depending on a part that is a treatment target or the type of treatment, for example.

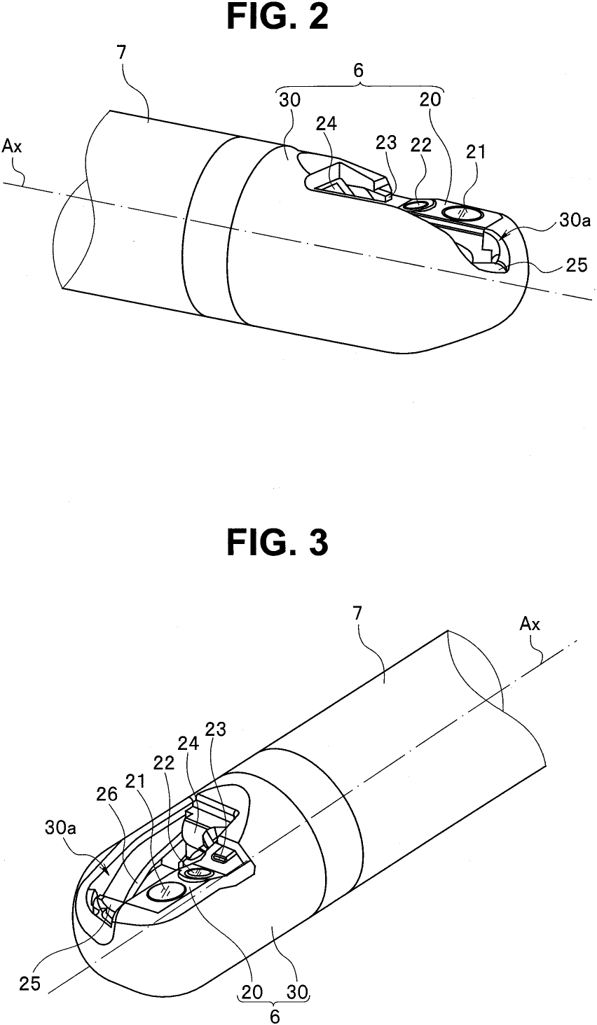

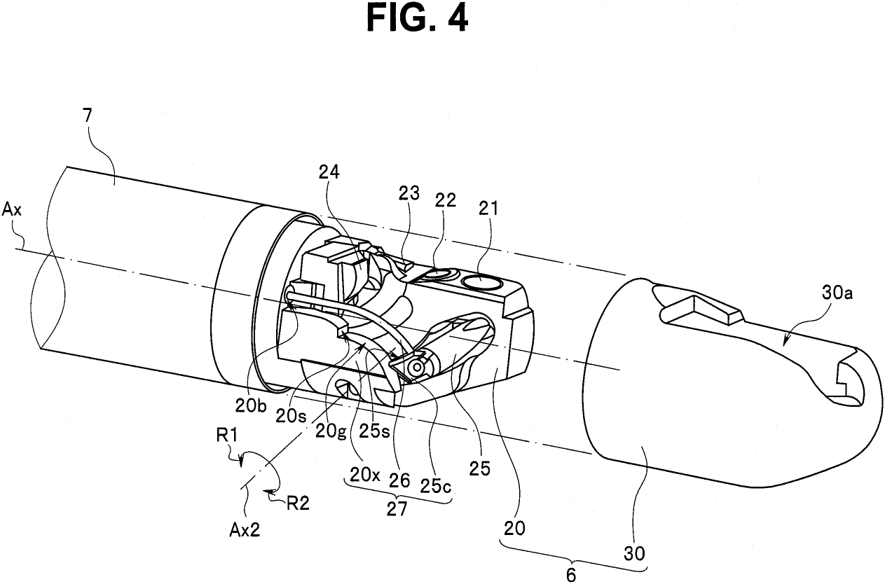

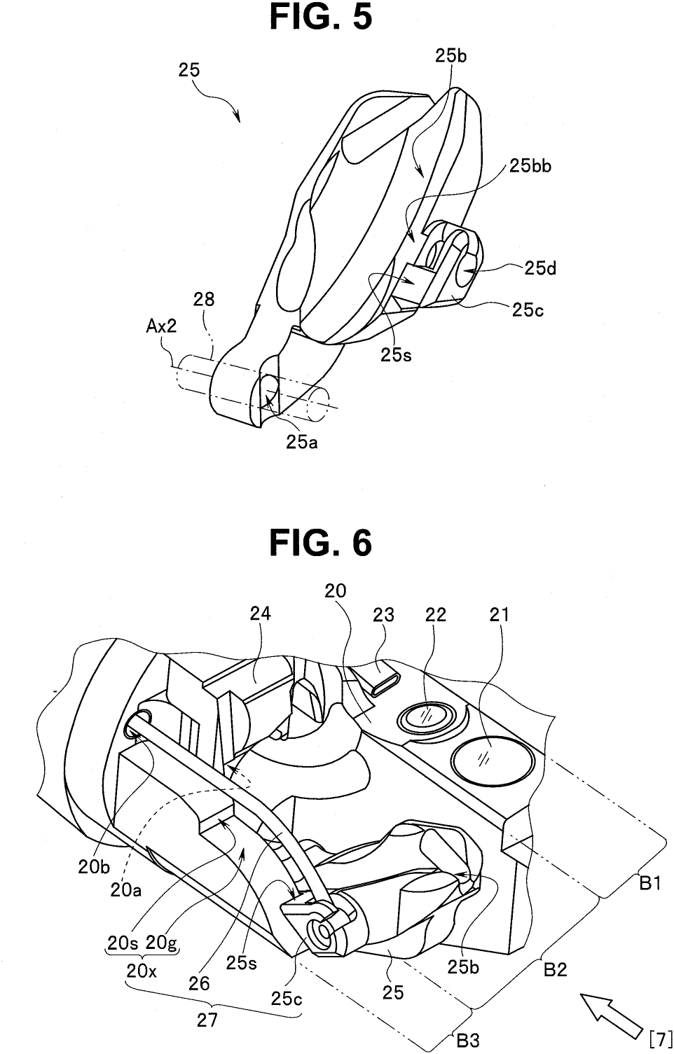

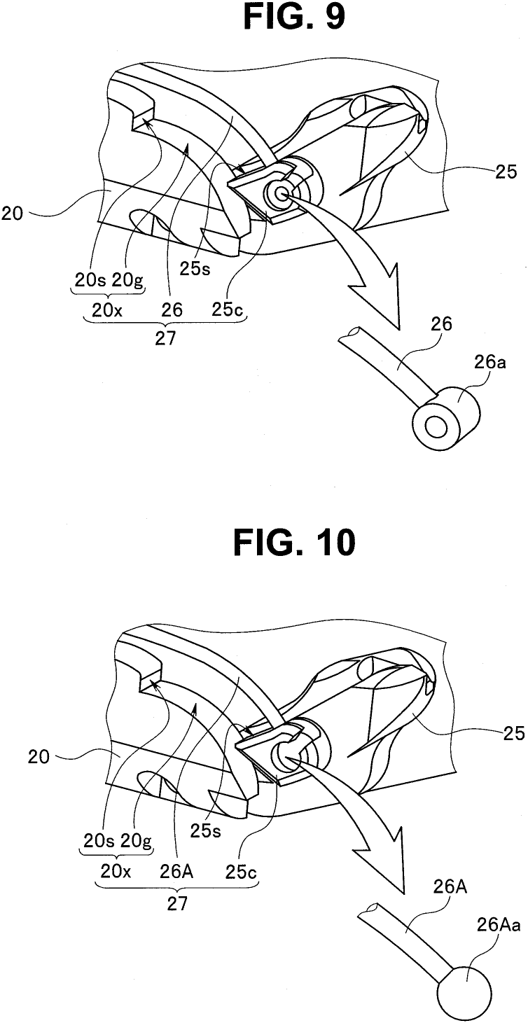

Next, a detailed configuration of the distal end structural portion 6 of the endoscope 2 of the present embodiment included in the endoscope system 1 configured in the above manner will be described with reference to FIGS. 2 to 10. FIGS. 2 and 3 are enlarged perspective views of main parts showing, in an enlarged manner, the distal end structural portion of the endoscope of the embodiment of the present disclosure. Of the drawings, FIG. 2 is a perspective view seen from a position obliquely on left of a distal end surface of the distal end structural portion. FIG. 3 is a perspective view seen from a position obliquely on right of the distal end surface of the distal end structural portion. FIG. 4 is an exploded perspective view showing a state where a distal end cover of the distal end structural portion is removed. FIG. 5 is a perspective view showing, in an enlarged manner, only the forceps raising base among internal structural members of the distal end structural portion. FIG. 6 is an enlarged perspective view showing a configuration of the forceps raising base and a forceps raising base operation mechanism. FIG. 7 is an enlarged plan view of the distal end structural portion seen in an arrow [7] direction in FIG. 6. FIG. 8 is a perspective view showing an internal structure of the distal end structural portion from which the distal end cover is removed, the internal structure being seen from a direction different from FIG. 4. FIG. 9 is an enlarged perspective view of main parts showing a structure for attaching an operation wire to a wire pulling portion. Like FIG. 9, FIG. 10 shows the structure for attaching the operation wire to the wire pulling portion, FIG. 10 showing a modification of a large diameter portion of the operation wire.

First, a schematic configuration of the distal end structural portion 6 of the endoscope 2 of the present embodiment will be described with reference to FIGS. 2 to 4. As illustrated, the distal end structural portion 6 includes a distal end structural portion main body 20, and a distal end cover 30. Note that in the following description, an axis line indicated by a reference sign Ax in FIGS. 2 and 3 will be referred to as an insertion axis of the insertion section 9 (the distal end structural portion 6). Furthermore, a direction along the insertion axis Ax will be referred to as a longitudinal direction of the insertion section 9.

The distal end structural portion main body 20 is a structural member provided at a distal end part of the insertion section 9. For example, the distal end structural portion main body 20 is formed using a metal material such as stainless steel. An illumination unit 21, an image pickup unit 22, a gas/liquid feeding nozzle 23, an insulation block 24, a forceps raising base (raising base) 25, a raising base operation mechanism section 27 (see FIG. 3 and the like; described in detail later) including a part of an operation wire (wire) 26, and the like are disposed on the distal end structural portion main body 20.

A region on the distal end structural portion main body 20 where the illumination unit 21, the image pickup unit 22, the gas/liquid feeding nozzle 23, and the like are disposed will be referred to as a first region. The first region is a region on the right when seen from a position facing a front surface of the distal end structural portion 6. In FIG. 6, a reference sign B1 indicates the first region. Furthermore, a region on the distal end structural portion main body 20 where the insulation block 24, the forceps raising base 25, and the like are disposed will be referred to as a second region. The second region is a region that is substantially a center part when seen from the position facing the front surface of the distal end structural portion 6. In FIG. 6, a reference sign B2 indicates the second region. Moreover, a region on the distal end structural portion main body 20 where the raising base operation mechanism section 27 including a part of the operation wire 26 is disposed will be referred to as a third region. The third region is a region on the left when seen from the position facing the front surface of the distal end structural portion 6. In FIG. 6, a reference sign B3 indicates the third region. Note that a wire pulling portion (attachment housing) 25c (described in detail later) that holds a distal end of the operation wire 26 is also disposed in the third region. The distal end portion 6 includes the first opening 20a connected to the instrument channel 17, a second opening (second distal-end-side opening) 20b connected to a wire channel, the wire 26 located in the wire channel and protruding distally from the second opening 20b, a first detent surface (rotation restriction portion) 20s, and the raising base 25 rotatable about an axis between a lowered position and a raised position. In the raised position, the raising base 25 intersects a projection of an axis of the instrument channel 17 extending from the first opening 20a. The raising base 25 includes the attachment housing 25c and a distal end of the wire 26 is attached to the raising base 25 at the attachment housing 25c. The wire 26 is movable within the wire channel to rotate the raising base 25 about the axis from the lowered position to the raised position. And in the raised position, a stop surface (abutting stopper surface) 25s of the attachment housing 25c contacts the first detent surface 20s to limit an amount of rotation of the raising base 25.

As described above, the illumination unit 21, the image pickup unit 22, the gas/liquid feeding nozzle 23 and the like are disposed in the first region B1 of the distal end structural portion main body 20. Of these, the illumination unit 21 includes the optical illumination member configured to receive the illumination light that is transmitted from the light source device 3 through the optical fiber cable or the like, and to radiate a range in a direction outside the distal end structural portion 6. Note that the configuration of the illumination unit 21 itself is the same as the configuration mounted in a conventional endoscope. Accordingly, a detailed description of the illumination unit 21 is omitted.

The image pickup unit 22 is a structural member including an image pickup optical system, an image pickup device, an image pickup device drive circuit, and the like. The image pickup optical system includes an optical lens that forms an optical image of an observation target object. The image pickup device is an electronic element configured to receive and photoelectrically convert the optical image that is formed by the image pickup optical system. The image pickup device is driven by the image pickup device drive circuit. The image pickup device drive circuit receives an instruction signal from the video processor 4 and drives the image pickup device. Image data obtained by photoelectric conversion by the image pickup device is transmitted to the video processor 4 via the image pickup device drive circuit. The video processor 4 performs image data processing on the image data that is received. Note that the configuration of the image pickup unit 22 itself is the same as the configuration mounted in a conventional endoscope. Accordingly, a detailed description of the image pickup unit 22 is omitted.

The gas/liquid feeding nozzle 23 is a structural member configured to discharge liquid or gas at a time of feeding liquid or gas to outside from the distal end structural portion 6. For example, when a liquid feeding operation is performed based on operation of an operation member among the plurality of operation members provided on the operation section 10, water for cleaning a surface of an observation window, in the image pickup optical system of the image pickup unit 22, that is exposed to outside is discharged from the gas/liquid feeding nozzle 23.

As described above, the insulation block 24, the forceps raising base 25, and the like are disposed in the second region B2 of the distal end structural portion main body 20. Moreover, the first distal-end-side opening 20a (see FIGS. 7 and 8) that is a first opening portion to which the treatment instrument insertion channel 17 is connected is formed in the second region B2.

As described above, the treatment instrument 40 is inserted from the forceps insertion port 11 and through the treatment instrument insertion channel 17. A distal end region (a distal end part) of the treatment instrument 40 protrudes outward from the first distal-end-side opening 20a (see FIGS. 7 and 8) of the distal end structural portion 6. At this time, a protruding direction of the distal end region of the treatment instrument 40 is changed through a raising operation on the forceps raising base 25 (described in detail later).

The insulation block 24 is a structural member that is interposed between the treatment instrument 40 and the distal end structural portion main body 20 when the treatment instrument 40 is raised by the forceps raising base 25 as described above. The insulation block 24 is formed using a ceramic material such as alumina. According to such a configuration, the insulation block 24 electrically disconnects the treatment instrument 40 and the distal end structural portion main body 20 from each other when the treatment instrument 40 is raised by the forceps raising base 25. At the same time, the insulation block 24 restricts movement of the treatment instrument 40 to beyond a maximum raised position.

In other words, in the process of raising of the treatment instrument 40 by the forceps raising base 25, the treatment instrument 40 abuts the insulation block 24, and turning of the forceps raising base 25 stops when the treatment instrument 40 is sandwiched between the forceps raising base 25 and the insulation block 24. Further movement of the treatment instrument 40 in a raising direction is thereby restricted.

The forceps raising base 25 is a structural member configured to change the protruding direction of the treatment instrument 40 protruding outward from the distal end structural portion 6 (the first distal-end-side opening 20a; see FIGS. 7 and 8). As described above, when the treatment instrument 40 is inserted from the forceps insertion port 11 and through the treatment instrument insertion channel 17, a part of the distal end protrudes outward from the first distal-end-side opening 20a of the distal end structural portion main body 20. When the forceps raising base 25 is turned in the raising direction when the treatment instrument 40 is in such a state, the treatment instrument 40 is raised together with the forceps raising base 25.

Now, the configuration of the forceps raising base 25 will be described in greater detail with reference to FIG. 5 and so on. The forceps raising base 25 is provided in a manner capable of rotating relative to the distal end structural portion 6. More specifically, the forceps raising base 25 is provided in a manner capable of rotating around a rotation axis Ax2 (see FIGS. 4 and 5) that may intersect with or be orthogonal to the insertion axis Ax (see FIGS. 2 to 4) of the distal end structural portion 6. An arrow sign R1 in FIG. 4 indicates the raising direction of the forceps raising base 25. An arrow sign R2 in FIG. 4 indicates a lowering direction of the forceps raising base 25. Moreover, in this case, rotation of the forceps raising base 25 is restricted to within a range (described in detail later). For example, the forceps raising base 25 is formed using a metal material such as stainless steel.

As shown in FIG. 5, a rotation center hole 25a, a treatment instrument guiding portion (deflecting surface) 25b, the wire pulling portion 25c, a wire engaging portion (seating fixture) 25d, an abutting stopper surface (stop surface) 25s, and the like are formed on the forceps raising base 25.

The rotation center hole 25a is a through hole where a support shaft member 28 is fixed, the support shaft member 28 being configured to pivotally support the forceps raising base 25 in a manner allowing rotation in a direction. The support shaft member 28 is disposed at a predetermined part of the distal end structural portion main body 20 in parallel to or offset the rotation axis Ax2. In the present embodiment, the support shaft member 28 is disposed extending across the first region B1 and the third region B3 (a distal end protrusion portion (guide body) 20x; described later) of the distal end structural portion main body 20, for example.

The treatment instrument guiding portion 25b is a contact surface that is formed to come into contact with the treatment instrument 40 protruding from the first distal-end-side opening 20a and to guide a proceeding direction of the treatment instrument 40 to a predetermined direction. For example, the treatment instrument guiding portion 25b is concave shaped in a cross-section in a direction may intersect with or be orthogonal to the insertion axis Ax. In this case, the concave shape of the treatment instrument guiding portion 25b has a larger diameter than a diameter of the treatment instrument 40 near a distal end portion. In this manner, with the endoscope 2, the protruding direction of the distal end part of the treatment instrument 40 inserted through the treatment instrument insertion channel 17 is guided by the forceps raising base 25 within a predetermined range. The raising base 25 can include the deflecting surface 25b and a side surface. In the raised position, the deflecting surface 25b can intersect the projection of the axis of the instrument channel 17 extending from the first opening 20a, and the side surface can include the attachment housing 25c. The attachment housing 25c can be integrated into a body of the raising base 25.

The wire pulling portion 25c is a structural part configured to hold the distal end of the operation wire 26. The wire pulling portion 25c includes a function of transmitting, to the forceps raising base 25, a pull force that is generated by a reciprocating movement (described in detail later) of the operation wire 26 along the insertion axis Ax. In this case, the operation wire 26 and the wire pulling portion 25c function as a part of the raising base operation mechanism section 27.

The wire pulling portion 25c is integrally formed with the forceps raising base 25. In this case, the wire pulling portion 25c and the treatment instrument guiding portion 25b of the forceps raising base 25 are formed including a surface where the wire pulling portion 25c and the treatment instrument guiding portion 25b are at least partially integrally joined to each other (see a reference sign 25bb in FIG. 5). The wire pulling portion 25c is formed at a position protruding to the third region B3 side, in a direction that may intersect with or be orthogonal to the insertion axis Ax (in a direction that may offset or be parallel to the rotation axis Ax2). In other words, the wire pulling portion 25c is formed at a position that is offset from the treatment instrument guiding portion 25b to the third region B3 side, in the direction may intersect with or be orthogonal to the insertion axis Ax (in the direction may offset or be parallel to the rotation axis Ax2).

Note that an example configuration where the wire pulling portion 25c is integrally formed with the forceps raising base 25 is described, but such an example configuration is not restrictive. For example, the wire pulling portion 25c that is formed separately from the forceps raising base 25 may be attached to a predetermined position on the forceps raising base 25.

The wire engaging portion 25d removably engages with a large diameter portion 26a (a so-called drum-like portion; a termination; see FIG. 9 described later) that is provided at the distal end of the operation wire 26. The wire engaging portion 25d causes the distal end of the operation wire 26 to be held by the wire pulling portion 25c by being engaged with the large diameter portion 26a. Then, when the operation wire 26 is pulled in a state where the large diameter portion 26a is engaged with the wire engaging portion 25d, the forceps raising base 25 is raised by the pull force that is transmitted from the large diameter portion 26a to the wire pulling portion 25c through the wire engaging portion 25d. Note that the large diameter portion 26a and the wire engaging portion 25d may be configured to be turnable relative to each other. The attachment housing 25c includes a seating fixture 25d. The distal end of the wire 26 can include the termination 26a. And the termination 26a can engage with the seating fixture 25d to attach the distal end of the wire 26 to the raising base 25. The distal end of the wire 26 can be detachably attached to the raising base 25 at the attachment housing 25c.

The abutting stopper surface 25s is an abutting surface that abuts a rotation restriction portion (first detent surface) 20s of the distal end protrusion portion 20x described later when the forceps raising base 25 rotates within a predetermined range where rotation is allowed. When the abutting stopper surface 25s abuts the rotation restriction portion 20s, rotation of the forceps raising base 25 in the raising direction is restricted. Note that a detailed configuration and operation of the distal end protrusion portion 20x including the rotation restriction portion 20s to which the abutting stopper surface 25s abuts will be described later.

The raising base operation mechanism section 27 including a part of the operation wire 26, and the like are disposed in the third region B3 of the distal end structural portion main body 20. Accordingly, a second distal-end-side opening 20b (see FIGS. 7 and 8) that is a second opening portion to which an operation wire insertion conduit (not shown) is connected is formed in the third region B3 of the distal end structural portion main body 20, the operation wire insertion conduit being a conduit that passes through the insertion section 9. According to such a configuration, a part of a distal end region of the operation wire 26 protrudes from the second distal-end-side opening 20b of the distal end structural portion main body 20 of the distal end structural portion 6, and the distal end of the operation wire 26 is held by the wire pulling portion 25c. The operation wire 26 performs the reciprocating movement in the direction along the insertion axis Ax according to an operation by an endoscope user.

A waterproof cover member, such as a cover tube, may be provided on the distal end region of the operation wire 26, or in other words, a region protruding from the second distal-end-side opening 20b, up to a position held by the wire pulling portion 25c. By providing such a cover tube or the like at least on a part of the operation wire 26 that is exposed to outside, a surface of the operation wire that is mainly formed from a metal material may be protected.

Note that a protruding length of the operation wire 26 from the second distal-end-side opening 20b is changed by a raising operation or a lowering operation. Accordingly, it is conceivable to form the cover tube or the like using a material that is capable of extending and contracting, or to form the cover tube or the like into an accordion shape. Furthermore, one end of the cover tube is sealed to a peripheral edge of the second distal-end-side opening 20b. In the same manner, the other end of the cover tube is sealed to a distal end portion of the operation wire 26. A waterproof function of the operation wire 26 may be secured by such a configuration.

Furthermore, the operation wire 26 is inserted inside the operation wire insertion conduit (not shown) that is provided inside the insertion section 9, between the distal end structural portion 6 and an operation member of the operation section 10. The distal end of the operation wire 26 partially protrudes outside from the second distal-end-side opening 20b of the distal end structural portion 6. As shown in FIG. 9, the distal end of the operation wire 26 includes the large diameter portion 26a where a diameter is increased. Moreover, the large diameter portion 26a engages with the wire pulling portion 25c. Furthermore, a proximal end of the operation wire 26 is connected to the operation member of the operation section 10 (not shown). According to such a configuration, when an operation force is received from the operation member of the operation section 10, the operation wire 26 performs the reciprocating movement inside the operation wire insertion conduit in the direction along the insertion axis Ax. A drive force by the reciprocating movement is transmitted to the forceps raising base 25 via the wire pulling portion 25c. The drive force transmitted to the forceps raising base 25 causes the forceps raising base 25 to rotate within a rotation range around the rotation axis Ax2.

Note that as an example configuration of the large diameter portion 26a of the operation wire 26, as shown in FIG. 9, the large diameter portion 26a may have a tube shape or a columnar shape. However, a mode of the large diameter portion of the operation wire is not limited to the example configuration shown in FIG. 9. For example, the large diameter portion of the operation wire may be configured as shown in FIG. 10. A distal end of an operation wire 26A shown in FIG. 10 includes a large diameter portion 26Aa may have a spherical shape, a ball shape, or a shape to engage the wire pulling portion 25c. Exactly the same function as the function of the large diameter portion 26a having the example configuration in FIG. 9 may be achieved by such a mode.

Furthermore, the wire pulling portion 25c is disposed in the third region B3 of the distal end structural portion main body 20, the wire pulling portion 25c being formed to protrude in the direction may intersect with or be orthogonal to the insertion axis Ax from a side surface of the forceps raising base 25.

Furthermore, the distal end protrusion portion 20x forming a part of the raising base operation mechanism section 27 is formed in the third region B3 of the distal end structural portion main body 20. The raising base operation mechanism section 27 here is a mechanism section including the distal end protrusion portion 20x, the wire pulling portion 25c, and the operation wire 26.

The distal end protrusion portion 20x extends more to a distal end side than the second distal-end-side opening 20b in the direction along the insertion axis Ax. The distal end protrusion portion 20x includes a guide surface 20g and the rotation restriction portion 20s. The guide surface 20g is formed from a curved surface for guiding the reciprocating movement of the operation wire 26 protruding from the second distal-end-side opening 20b. The curved surface of the guide surface 20g is formed along a trajectory by which, when the forceps raising base 25 is in a lowered state, the operation wire 26 connects to the wire pulling portion 25c after extending from the second distal-end-side opening 20b. The distal end portion can include the guide surface 20g. The guide surface 20g is located distally relative to the first detent surface 20s, and a portion of the attachment housing 25c travels over the guide surface 20g during rotation of the raising base 25. The attachment housing 25c can be with contact or without contact the guide surface 20g during the rotation. The guide surface 20g extends from the first detent surface 20s in a direction of a longitudinal axis of the insertion section 9, and the guide surface 20g can include a curved surface portion. The curved surface portion can curve downward relative to the longitudinal axis of the insertion section 9. The guide surface 20g can include a proximal end surface portion, and the first detent surface 20s and the proximal end surface portion may intersect at a right angle or at an acute angle.

The rotation restriction portion 20s is provided on one end portion of the guide surface 20g, close to a proximal end. The rotation restriction portion 20s is provided to restrict a rotation angle by which the wire pulling portion 25c may be rotated together with the forceps raising base 25 by the pull force from the operation wire 26. In other words, the rotation restriction portion 20s restricts a rotation angle of the forceps raising base 25 in the raising direction by abutting the abutting stopper surface 25s of the wire pulling portion 25c that rotates together with the forceps raising base 25 within a range of a rotation angle.

Note that the curved surface of the guide surface 20g is formed with a gap to a movement traj ectory within a movement range of rotation of the wire pulling portion 25c that is rotated together with the forceps raising base 25 by the pull force from the operation wire 26. The gap is provided to prevent the wire pulling portion 25c from coming into contact with parts other than the rotation restriction portion 20s.

According to such a configuration, of structural members provided in the third region B3 of the distal end structural portion main body 20, the second distal-end-side opening 20b, the guide surface 20g, the rotation restriction portion 20s, and the wire pulling portion 25c are disposed on a straight line that may offset or be parallel to a line extending along the insertion axis Ax. In other words, a part of the operation wire 26 protruding from the second distal-end-side opening 20b, the distal end protrusion portion 20x including the guide surface 20g and the rotation restriction portion 20s, and the wire pulling portion 25c are disposed on the straight line that may offset or be parallel to the line extending along the insertion axis Ax. In other words, the raising base operation mechanism section 27 formed from the operation wire 26, the distal end protrusion portion 20x, and the wire pulling portion 25c is provided in a region outside a treatment instrument guide range. The treatment instrument guide range here is a range where the treatment instrument 40 may be moved in up/down/left/right directions when the forceps raising base 25 is raised, as seen from a distal end side along the insertion axis Ax. For example, as shown in FIG. 7, a region indicated by a reference sign [A] that is seen from a position facing a front surface of the distal end structural portion 6 corresponds to the treatment instrument guide range.

Furthermore, the distal end protrusion portion 20x (the third region B3) and the forceps raising base 25 (the second region B2) are disposed in respective regions of the distal end structural portion main body 20 that are adjacent to each other. In this case, a surface of the distal end protrusion portion 20x facing the forceps raising base 25 is smoothly formed in a range of a rotation operation of the forceps raising base 25.

Furthermore, a gap is provided at a part where the distal end protrusion portion 20x and the forceps raising base 25 face each other. The gap in this case is set smaller than a dimension of the first distal-end-side opening 20a even at a largest part. This is to prevent the treatment instrument 40 from being getting caught between the distal end protrusion portion 20x and the forceps raising base 25. At the same time, by providing the gap that is smaller than the dimension of the first distal-end-side opening 20a, the treatment instrument 40 can be expected to be guided to the treatment instrument guiding portion 25b side of the forceps raising base 25. The guide surface 20g may be on a guide body 20x extending distally relative to the second opening 20b, and the guide body 20x further can include a first side surface oriented toward a space between the raising base 25 and the first opening 20a. The rotation of the raising base 25 can occur along a first rotation path, the first opening has a first diameter, and the first side surface of the guide body 20x can be separated from the first rotation path by a gap. A length of the gap is smaller than a length of the first diameter. The first detent surface 20s can include a flat surface portion, the flat surface portion is normal to a longitudinal direction of the insertion section 9.

Moreover, the first distal-end-side opening 20a and the second distal-end-side opening 20b are disposed next to each other in the direction that may intersect with or be orthogonal to the insertion axis Ax, or in other words, in a width direction of the distal end structural portion main body 20 (see FIG. 7). However, in this case, the first distal-end-side opening 20a and the second distal-end-side opening 20b are formed at positions that are shifted from or offset each other in the direction along the insertion axis Ax. The distal end portion 6 has a central axis extending longitudinally from a proximal end to a distal end. In a cross-section perpendicular to the central axis: (i) the first detent surface is at a first distance from the axis of the instrument channel 17, (ii) the second opening 20b is at a second distance from the axis of the instrument channel 17, (iii) the attachment housing 25c is at a third distance from the axis of the instrument channel 17, and (iv), a radius of the instrument channel 17 has a radial length. The first distance, the second distance, and the third distance can be each larger than the radial length. The first detent surface 20s restricts a movement of the raising base 25 in a proximal direction.

The distal end cover 30 is a cover member that is attached to a distal end side of the distal end structural portion main body 20. The distal end cover 30 at least covers a part of the forceps raising base 25, the operation wire 26, the wire pulling portion 25c, and an outer surface of the distal end protrusion portion 20x. Furthermore, an opening 30a for not obstructing the forceps raising base 25 moving in the raising direction by rotation is provided in the distal end cover 30 (see FIGS. 2 to 4). According to such a configuration, the forceps raising base 25 operates without coming into contact with the distal end cover 30 at a time of rotation of the forceps raising base 25 under the pull force from the operation wire 26. The distal end cover 30 is attached to the distal end portion 6.

The configuration of the endoscope 2 of the present embodiment is as described above. Operation of the forceps raising base 25 of the distal end structural portion 6 of the endoscope 2 configured in the above manner will be described below with reference to FIGS. 3, 4, and 11 to 16.

The forceps raising base 25 is lowered when the pull force from the operation wire 26 is not applied to the forceps raising base 25. The state at this time will be referred to as a lowered state of the forceps raising base 25 (see FIGS. 2 to 4, FIG. 6, FIGS. 8 to 10, and FIG. 15).

When the pull force from the operation wire 26 is applied to the forceps raising base 25, the forceps raising base 25 is rotated and raised. The state at this time will be referred to as a raised state of the forceps raising base 25 (see FIGS. 11 to 14, and FIG. 16).

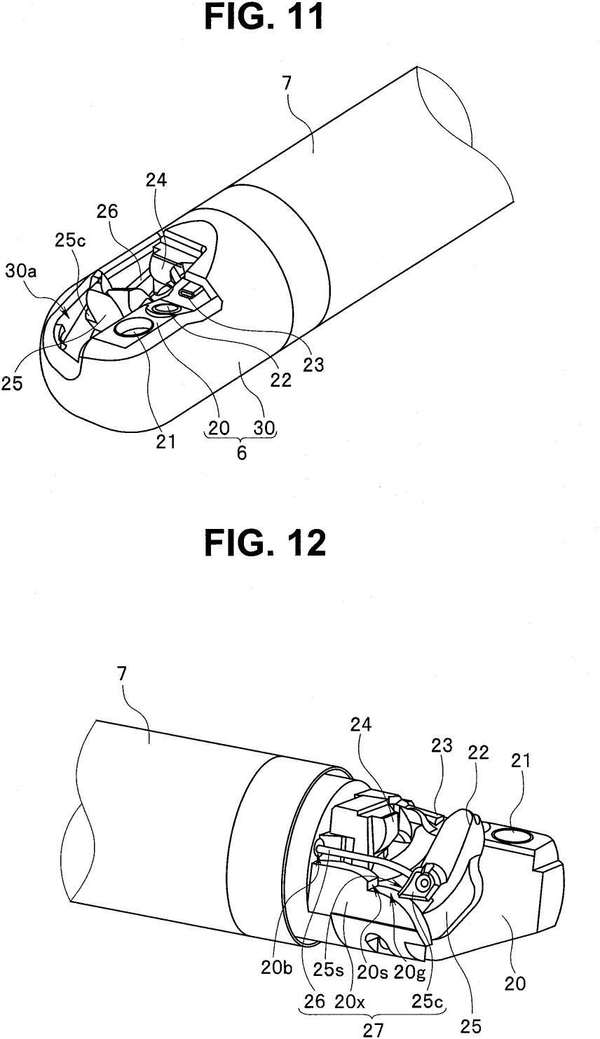

Like FIG. 3, FIG. 11 is a diagram showing the distal end structural portion. Furthermore, FIG. 12 shows a state where the distal end cover is removed from the distal end structural portion in FIG. 11. FIGS. 11 and 12 show a state where the forceps raising base is raised halfway.

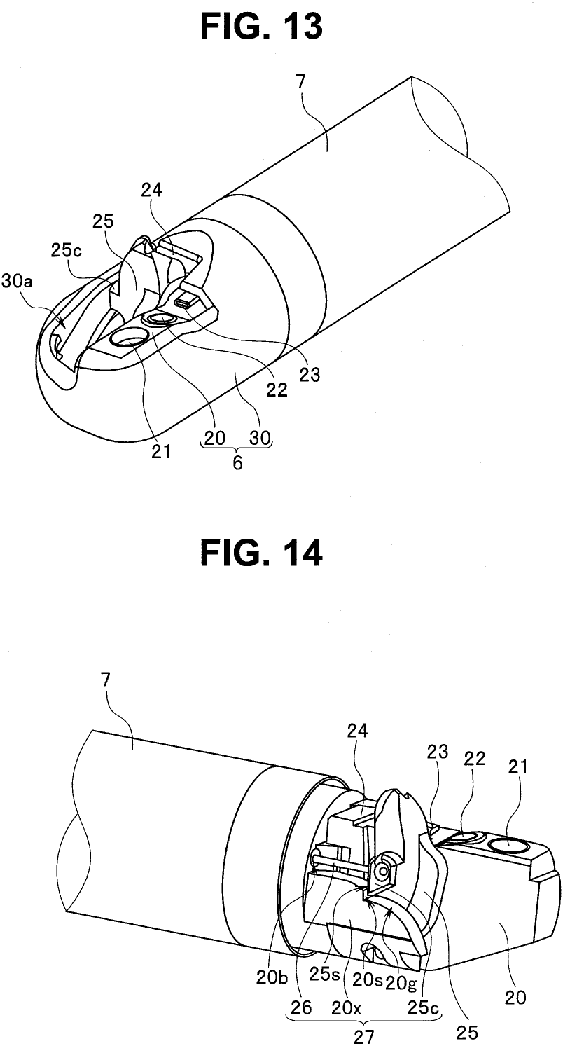

Like FIGS. 3 and 11, FIG. 13 is a diagram showing the distal end structural portion. Furthermore, FIG. 14 shows a state where the distal end cover is removed from the distal end structural portion in FIG. 13. FIGS. 13 and 14 show a maximum raised state of the forceps raising base.

Note that FIG. 3, FIG. 4, and FIGS. 11 to 14 show a state where the treatment instrument is not included so as to describe operation of the forceps raising base itself.

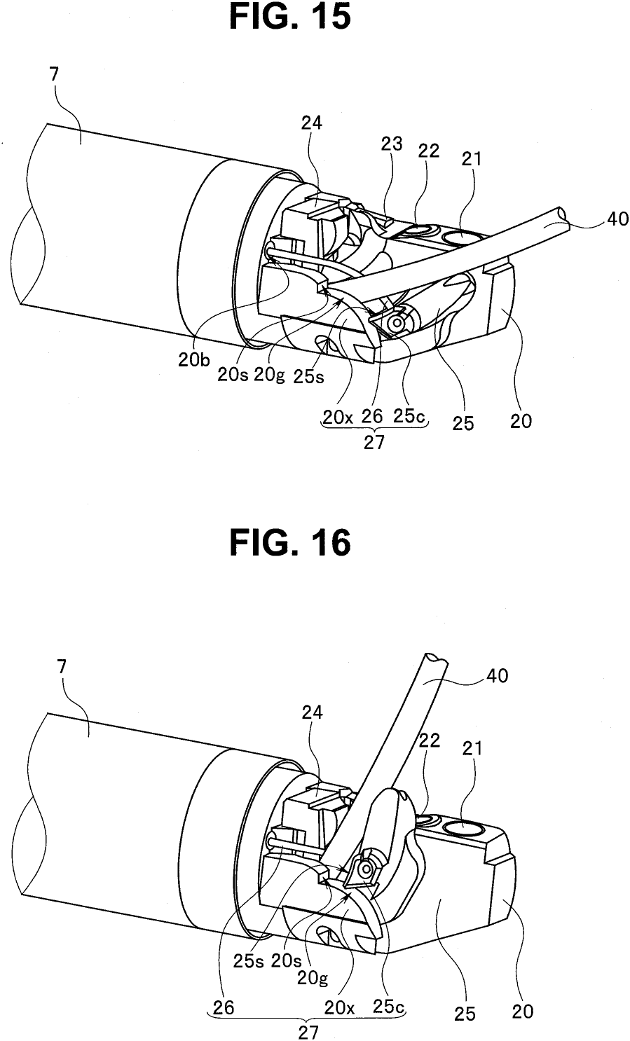

FIGS. 15 and 16 show the treatment instrument that is raised by operation of the forceps raising base. Of the drawings, FIG. 15 shows the distal end structural portion from which the distal end cover is removed, and shows a state where the treatment instrument is included. Furthermore, FIG. 16 shows a state after the state in FIG. 15, where the forceps raising base is raised together with the treatment instrument and the treatment instrument is at a maximum raised position.

With the endoscope 2 of the present embodiment, first, in a state where the forceps raising base 25 is not operated, the forceps raising base 25 is in the lowered state shown in FIGS. 3 and 4. At this time, the reciprocating movement of the operation wire 26 is stopped. In other words, the pull force from the operation wire 26 is not transmitted to the wire pulling portion 25c of the forceps raising base 25. At this time, a rotational biasing force in a lowering direction R2 is applied to the forceps raising base 25. However, rotation of the forceps raising base 25 in the lowering direction R2 by the rotational biasing force is restricted due to the forceps raising base 25 abutting a part on an inner surface of the distal end cover 30. The lowered state in FIGS. 3 and 4 is thereby maintained.

It is assumed that the raising operation is performed on the forceps raising base 25 in the lowered state shown in FIGS. 3 and 4, by operation of the operation member of the operation section 10 by an endoscope user.

In response to the raising operation at this time, the operation wire 26 is pulled toward the operation section 10 side in the direction along the insertion axis Ax. The pull force from the operation wire 26 is transmitted to the forceps raising base 25 via the wire pulling portion 25c. The forceps raising base 25 thus rotates in a raising direction R1 around the rotation axis Ax2 from the lowered state in FIGS. 3 and 4. For example, the forceps raising base 25 is displaced to the state in FIGS. 11 and 12. At this time, during movement of the forceps raising base 25 in the raising direction, the wire pulling portion 25c moves along the curved surface of the guide surface 20g of the distal end protrusion portion 20x without abutting the guide surface 20g and while maintaining a gap to the guide surface 20g.

When the raising operation of the forceps raising bases 25 is continued in this state and the operation wire 26 keeps being pulled, at the end, the abutting stopper surface 25s of the wire pulling portion 25c of the forceps raising base 25 abuts the rotation restriction portion 20s of the distal end protrusion portion 20x. Rotation of the forceps raising base 25 in the raising direction R1 is thereby restricted. The state of the forceps raising base 25 at this time is the maximum raised state shown in FIGS. 13 and 14.

As described above, here, rotation of the forceps raising base 25 is restricted due to the abutting stopper surface 25s abutting the rotation restriction portion 20s. Accordingly, the forceps raising base 25 does not rotate in the raising direction R1 beyond a position shown in FIGS. 13 and 14.

Note that the wire pulling portion 25c of the forceps raising base 25 does not come into contact with parts other than the rotation restriction portion 20s during movement from the lowered state shown in FIGS. 3 and 4 to the maximum raised state shown in FIGS. 13 and 14. The forceps raising base 25 may thus move smoothly within a rotation range.

Then, in the maximum raised state in FIGS. 13 and 14, the endoscope user operates the operation member of the operation section 10 and relaxes the pull force from the operation wire 26. The lowering operation may thus be performed on the forceps raising base 25.

As described above, with the forceps raising base 25, a raised position of the forceps raising base 25 may be freely set in the range (in the range of the rotation angle) by causing the operation wire 26 to perform the reciprocating movement, by performing the raising operation of pulling the operation wire 26 and the lowering operation of slackening the operation wire 26 as appropriate.

Note that in the course of a reciprocating movement operation on the operation wire 26, the endoscope user may maintain a stopped position of the operation wire 26 by operating a different operation member of the operation section 10. Any stopped position within the movement range may thus be maintained with respect to the forceps raising base 25.

The operation at the time of operating the forceps raising base 25 in a state where the treatment instrument 40 is inserted through the treatment instrument insertion channel 17 of the insertion section 9 of the endoscope 2 of the present embodiment is as follows.

First, the treatment instrument 40 is inserted from the forceps insertion port 11 and through the treatment instrument insertion channel 17. Then, a distal end part of the treatment instrument 40 is placed in a state where the distal end part protrudes from the first distal-end-side opening 20a of the distal end structural portion main body 20. At this time, the forceps raising base 25 is assumed to be in the lowered state. The state at this time is shown in FIG. 15.

The endoscope user operates the operation member of the operation section 10 in the state in FIG. 15, and performs the raising operation on the forceps raising base 25. Then, in response to the raising operation, the operation wire 26 is pulled toward the operation section 10 side in the direction along the insertion axis Ax. The pull force from the operation wire 26 causes the forceps raising base 25 to be raised via the wire pulling portion 25c. The forceps raising base 25 thereby rotates in the raising direction R1 around the rotation axis Ax2. At this time, the distal end part of the treatment instrument 40 is raised following rotation of the forceps raising base 25 in the raising direction R1. The protruding direction of the treatment instrument 40 is thus changed.

Then, when the raising operation on the forceps raising base 25 is continued, at the end, the treatment instrument 40 abuts the insulation block 24. Movement of the treatment instrument 40 in the raising direction and rotation of the forceps raising base 25 in the raising direction R1 are thereby restricted. The state of the treatment instrument 40 and the forceps raising base 25 at this time is the state shown in FIG. 16. At this time, the treatment instrument 40 cannot be moved in the raising direction beyond the position shown in FIG. 16. Furthermore, the forceps raising base 25 does not rotate in the raising direction beyond the position shown in FIG. 16. Note that in this state, the abutting stopper surface 25s of the forceps raising base 25 and the rotation restriction portion 20s of the distal end protrusion portion 20x do not abut against each other.

As described above, when the treatment instrument 40 is inserted in the insertion section 9 and the distal end part of the treatment instrument 40 protrudes from the distal end structural portion main body 20, the treatment instrument 40 that is raised by the forceps raising base 25 abuts the insulation block 24, and the movement range of the treatment instrument 40 is thus restricted.

As described above, according to the embodiment, with the endoscope 2 including the forceps raising base 25 configured to change the protruding direction of the treatment instrument 40 protruding outward from the first distal-end-side opening 20a of the distal end structural portion 6 of the insertion section 9, the forceps raising base 25 includes the wire pulling portion 25c that is formed protruding in the direction that may intersect with or be orthogonal to the insertion axis Ax from the side surface. Furthermore, the distal end protrusion portion of the distal end structural portion 6 includes the guide surface 20g that guides the reciprocating movement of the operation wire 26, and the rotation restriction portion 20s that restricts the rotation angle by which the wire pulling portion 25c is able to rotate together with the forceps raising base 25. The rotation restriction portion 20s is provided at an end portion of the guide surface 20g.

According to such a configuration, at the distal end structural portion 6 of the endoscope 2 of the present embodiment, the rotation restriction portion 20s that restricts rotation of the forceps raising base 25 may be provided at a position outside the treatment instrument guide range at the distal end structural portion main body 20.

Accordingly, with such a configuration, the treatment instrument 40 does not get caught by the rotation restriction portion 20s or the like at the time of insertion/removal of the treatment instrument 40, for example. Accordingly, operability may be secured at all times for the endoscope 2 and the treatment instrument 40.

Furthermore, in the embodiment described above, a partitioning wall is not provided between the forceps raising base and the forceps raising base operation mechanism, and a mechanism is adopted where the forceps raising base and the operation wire are connected and the forceps raising base is directly pulled. According to such a configuration, configurations of parts may be more simplified and the number of parts may be reduced compared to other conventional configurations, or in other words, a case of adopting a configuration where the forceps raising base and the mechanism section for operating the forceps raising base are disposed in respective spaces partitioned by a wall member or the like. Accordingly, manufacturing costs of the distal end structural portion main body 20, assembly of each structural member, and the like may be reduced.

Furthermore, the wire pulling portion 25c is integrally formed in a manner protruding on a side of the forceps raising base 25, and also, the second distal-end-side opening 20b, the guide surface 20g, the rotation restriction portion 20s, and the wire pulling portion 25c are each provided on a straight line extending along the insertion axis Ax. According to such a configuration, the operation wire 26 protruding from the second distal-end-side opening 20b extends linearly without being curved and is held by the wire pulling portion 25c. Accordingly, bending or the like of the operation wire 26 of a conventional configuration becomes unnecessary and ease of assembly is increased, and also, durability of the operation wire 26 is increased.

The wire pulling portion 25c is formed including a surface that is integrally joined to the treatment instrument guiding portion 25b of the forceps raising base 25. According to such a configuration, the treatment instrument 40 does not get caught by a structural part such as the wire pulling portion 25c at the time of insertion/removal of the treatment instrument 40, for example. Accordingly, there is an advantageous effect that reduction in operability of the endoscope 2 and the treatment instrument 40 may be prevented.

The guide surface 20g of the distal end protrusion portion 20x is formed as a curved surface, and the curved surface is formed along the trajectory of the operation wire 26 protruding and extending forward from the second distal-end-side opening 20b. According to such a configuration, the operation wire 26 is able to smoothly rotate the forceps raising base 25 without coming into contact with the distal end protrusion portion 20x (the curved surface).

Moreover, the curved surface is formed with a gap to the wire pulling portion 25c. According to such a configuration, the wire pulling portion 25c moving according to rotation of the forceps raising base 25 is prevented from coming into contact with parts other than the rotation restriction portion 20s in the movement range. According to such a configuration, smooth rotation of the forceps raising base 25 may be maintained.

With the distal end structural portion of the endoscope of the embodiment described above, rotation in the lowered state of the forceps raising base is restricted by the forceps raising base abutting a part on the inner surface of the distal end cover.

A modification of the distal end structural portion of the endoscope of the present embodiment described next illustrates an example configuration where the rotation restriction portion in the lowering direction of the forceps raising base is provided on the distal end structural portion. FIG. 17 is a diagram showing a modification of the distal end structural portion of the endoscope of the embodiment of the present disclosure. Note that, like FIG. 4 and the like, FIG. 17 shows the state where the distal end cover is removed.

A basic configuration of the distal end structural portion of the modification shown in FIG. 17 includes same configuration as the configuration of the embodiment described above. In the present modification, configurations of a distal end protrusion portion 20Bx provided on a distal end structural portion main body 20B, and a wire pulling portion 25Bc provided on a forceps raising base 25B are slightly different from configurations of the distal end structural portion of the embodiment described above. Accordingly, components that are the same as the components of the embodiment described above will be denoted by same reference signs and detailed description will be omitted, and only different parts will be described below in detail.

As shown in FIG. 17, like the distal end protrusion portion 20x according to the embodiment described above, the distal end protrusion portion 20Bx provided on the distal end structural portion main body 20B of the distal end structural portion according to the present modification protrudes more to the distal end side than the second distal-end-side opening 20b in the direction along the insertion axis Ax.

The distal end protrusion portion 20Bx includes the guide surface 20g, a first rotation restriction portion 20sa (a first abutting portion or first detent surface), and a second rotation restriction portion 20sb (a second abutting portion or second detent surface). As in the embodiment described above, the guide surface 20g includes a curved surface that guides the reciprocating movement of the operation wire 26 protruding from the second distal-end-side opening 20b.

The first rotation restriction portion 20sa is provided on one end portion of the guide surface 20g, close to the proximal end. In other words, the first rotation restriction portion 20sa corresponds to the rotation restriction portion 20s of the embodiment described above. Accordingly, when abutted by a part of the wire pulling portion 25Bc (a first part; a first abutting stopper surface 25sa) that rotates together with the forceps raising base 25B, the first rotation restriction portion 20sa restricts a rotation angle of the forceps raising base 25B in the raising direction. In other words, when the forceps raising base 25B is in the maximum raised state, the first rotation restriction portion 20sa (the first abutting portion) restricts rotation of the forceps raising base 25B in the raising direction by being abutted by the first abutting stopper surface 25sa (the first part) of the wire pulling portion 25Bc.

The second rotation restriction portion 20sb is provided on the other end portion of the guide surface 20g, close to the distal end. When abutted by a part of the wire pulling portion 25Bc (a second part; a second abutting stopper surface 25sb) that rotates together with the forceps raising base 25B, the second rotation restriction portion 20sb restricts the rotation angle of the forceps raising base 25B in the lowering direction. In other words, when the forceps raising base 25B is in the lowered state, the second rotation restriction portion 20sb (the second abutting portion) restricts rotation of the forceps raising base 25B in the lowering direction by being abutted by the second abutting stopper surface 25sb (the second part) of the wire pulling portion 25Bc. The distal end portion 6 can include the second detent surface 20sb, in the lowered position, the attachment housing 25c contacts the second detent surface 20sb. The first detent surface 20sa restricts movement of the raising base 25 in a first rotation direction, and the second detent surface 20sb restricts movement of the raising base 25 in a second rotation direction. The first rotation direction is opposite to the second rotation direction. The guide surface 20g can extend between the first detent surface 20sa and the second detent surface 20sb, and a portion of the attachment housing 25c travels over contacts the guide surface 20g during rotation of the raising base 25. The attachment housing 25c can be with contact or without contact the guide surface 20g during the rotation.

In accordance with the distal end protrusion portion 20Bx, the first abutting stopper surface 25sa (the first part) and the second abutting stopper surface 25sb (the second part) of the wire pulling portion 25Bc of the forceps raising base 25B of the distal end structural portion of the present modification are formed at different positions.

The first abutting stopper surface 25sa here corresponds to the abutting stopper surface 25s of the embodiment described above. Accordingly, when the forceps raising base 25B moves in the raising direction, the first abutting stopper surface 25sa abuts the first rotation restriction portion 20sa to thereby restrict rotation of the forceps raising base 25B in the raising direction.

Furthermore, when the forceps raising base 25B moves in the lowering direction, the second abutting stopper surface 25sb abuts the second rotation restriction portion 20sb to thereby restrict rotation of the forceps raising base 25B in the lowering direction. Other configurations are the same as the configurations of the embodiment described above.

With the present modification configured in the above manner, a mechanism configured to restrict rotation of the forceps raising base 25B in the lowering direction (the second rotation restriction portion 20sb and the second abutting stopper surface 25sb) is further included. According to such a configuration, rotation of the forceps raising base 25B in both the raising direction and the lowering direction may be reliably restricted. Accordingly, the movement range of the forceps raising base 25B may be reliably set.

The endoscope illustrated in the embodiment described above may be applied to a single-use endoscope that is disposed of after being used once, in addition to a so-called reusable endoscope that can be repeatedly used by being subjected to cleaning/sterilization after being used, for example. In the case of application to a single-use endoscope, the manufacturing cost may be expected to be further reduced. The endoscope 2 can be applied to, for example, a single-use endoscope to be disposed of after being used once but may be a re-use endoscope to be repeatedly used.

As described above, with the distal end structural portion of the endoscope of the embodiment described above, at the time of performing a predetermined treatment by inserting the treatment instrument in the insertion section, the distal end of the treatment instrument is caused to protrude outward from the first distal-end-side opening of the distal end structural portion.

At this time, depending on a bent state of the insertion section of the endoscope, for example, the distal end of the treatment instrument may slip into a gap between the forceps raising base or the operation wire and the inner surface of the distal end cover due to the distal end of the treatment instrument moving in a direction away from a guiding surface of the forceps raising base.

In such a case, when the treatment instrument gets caught inside the distal end structural portion, the treatment instrument is prevented from moving further forward. In such a state, an unnecessary task of removing the treatment instrument and inserting the treatment instrument again, for example, has to be performed, and operability of the endoscope and the treatment instrument is reduced.

There are various types of treatment instruments to be used in combination with such an endoscope. The treatment instrument is configured to be able to smoothly move forward and backward near the forceps raising base at all times regardless of the type of the treatment instrument.

Accordingly, with the distal end structural portion 6 of the endoscope 2 of the present embodiment, an inner shape of the distal end cover 30 is improved, and a configuration is achieved with which the treatment instrument 40 is able to smoothly move forward and backward at all times without getting caught near the forceps raising base 25.

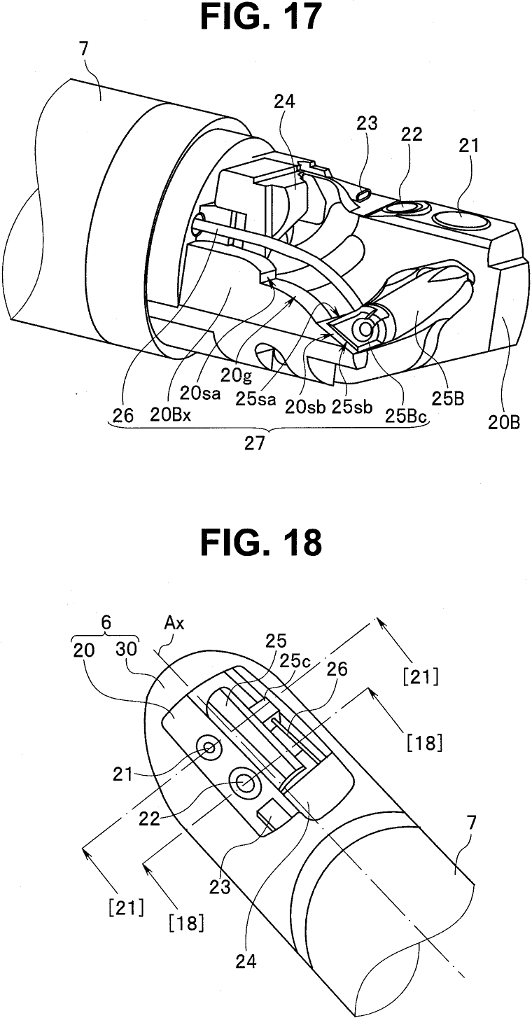

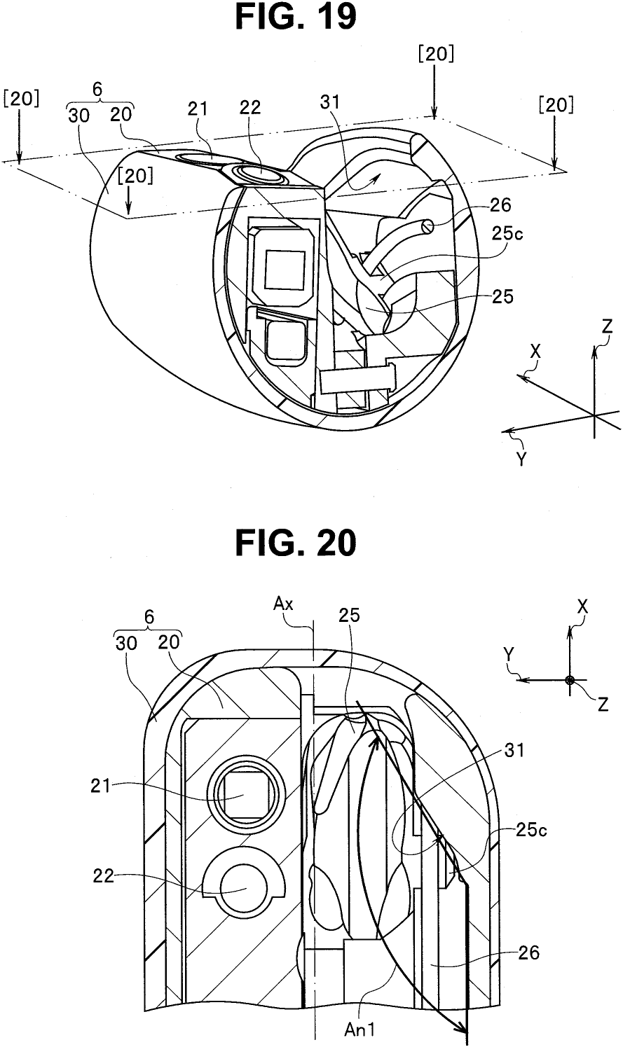

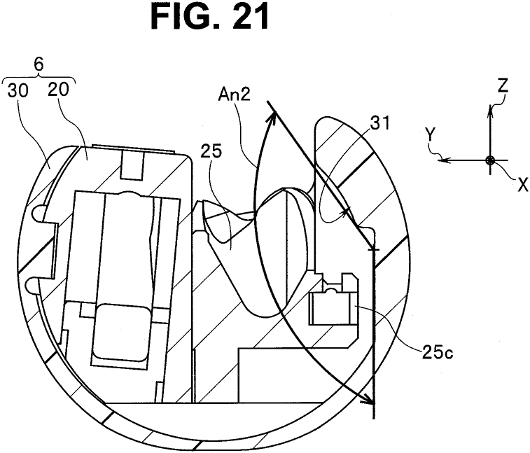

FIGS. 18 to 21 are diagrams schematically showing the distal end structural portion of the endoscope of the embodiment of the present disclosure. Of the drawings, FIG. 18 is a diagram schematically showing an external appearance of the distal end structural portion of the endoscope of the present embodiment. FIG. 19 shows a cross-sectional surface along a line [18]-[18] in FIG. 18. FIG. 20 shows a cross-sectional surface along a surface indicated by arrows [20] in FIG. 19. FIG. 21 shows a cross-sectional surface along a line [21]-[21] in FIG. 18. Here, with respect to the distal end structural portion 6, the direction along the insertion axis Ax will be defined as an X-axis, and two axes that may intersect with or be orthogonal to the X-axis will be defined as a Y-axis and a Z-axis, respectively. Of the axes, the Y-axis indicates a width direction of the distal end structural portion 6, and the Z-axis indicates an up-down direction of the distal end structural portion 6 (see FIGS. 19 to 21).

As described above, the distal end structural portion 6 of the insertion section 9 of the endoscope 2 is formed from the distal end structural portion main body 20 and the distal end cover 30. Of the two, the distal end structural portion main body 20, and each structural member that is attached to the distal end structural portion main body 20 are as described above. Furthermore, as described above, the distal end cover 30 is a cover member that is attached on the distal end side of the distal end structural portion main body 20. The distal end cover 30 here at least covers a part of the forceps raising base 25, the operation wire 26, the wire pulling portion 25c, and the outer surface of the distal end protrusion portion 20x. Furthermore, the opening 30a that enables the raised state at the time of rotation of the forceps raising base 25 is provided in the distal end cover 30.

In relation to the inner surface of the distal end cover 30, a treatment instrument guiding surface 31 that is a smooth curved surface that is continuously joined to the opening 30a of the distal end cover 30 is formed on a side wall surface near the forceps raising base 25.

As shown in FIG. 20, for example, the treatment instrument guiding surface 31 here is set such that an angle An1 of the side wall surface of the distal end cover 30 relative to an X-axis direction (in other words, a direction of movement of the treatment instrument 40 toward the distal end) is an obtuse angle of about 150 degrees.

Furthermore, as shown in FIG. 21, for example, the treatment instrument guiding surface 31 is set such that an angle An2 of the side wall surface of the distal end cover 30 relative to a Z-axis direction that may intersect with or be orthogonal to the X-axis (in other words, a direction in which the treatment instrument 40 is raised) is an obtuse angle of about 145 degrees.

By forming the treatment instrument guiding surface 31 according to such settings on the inner surface of the distal end cover 30, the distal end of the treatment instrument 40 may be smoothly guided in a direction of the opening 30a of the distal end cover 30 at a time when the treatment instrument 40 is caused to move forward toward the distal end side with the forceps raising base 25 in the lowered state or at a time of raising the treatment instrument 40 by the forceps raising base 25.

Therefore, according to such a configuration, the treatment instrument 40 may smoothly move forward and backward at all times at the distal end structural portion 6 of the endoscope 2 of the present embodiment without getting caught near the forceps raising base 25. Furthermore, also at the time of insertion/removal of the treatment instrument 40, the treatment instrument 40 may be smoothly inserted or removed at all times without the treatment instrument 40 getting caught inside the distal end cover 30.

Furthermore, for example, even in a case where solid residue or the like inside the body cavity enters inside the distal end structural portion 6 from the opening 30a of the distal end cover 30 or the like during use of the endoscope 2, because the inner surface of the distal end cover 30 is formed into a smooth shape, such solid residue or the like may be easily discharged outside.

Furthermore, not only the inner shape of the distal end cover 30, but also a part between the inner surface of the distal end cover 30 and the treatment instrument guiding portion 25b of the forceps raising base 25 may be formed to connect the parts with a smooth curved surface. Also in this case, the same advantageous effects may be obtained.

The present disclosure is not limited to the embodiment described above, and various modifications and applications may, of course, be made within the scope of the gist of the disclosure. Furthermore, the embodiment described above includes disclosures at various stages, and various disclosures may be extracted by combining a plurality of disclosed structural conditions as appropriate. For example, in the case where a problem to be solved by the disclosure may be solved and advantageous effects of the disclosure may be obtained even when some of the structural conditions are removed from all the structural conditions indicated in the embodiment described above, a configuration excluding such structural conditions may be extracted as a disclosure. Moreover, structural elements of different embodiments may be combined as appropriate. The disclosure is not limited by any specific embodiment, and is limited only by the appended claims.

Example 1. An endoscope comprising:
a distal end structural portion provided at a distal end part of an insertion section configured to be inserted into a subject;
a forceps raising base provided in a manner capable of turning relative to the distal end structural portion so as to raise a treatment instrument protruding from a first opening portion provided in the distal end structural portion;
an operation wire protruding from a second opening portion provided in the distal end structural portion, the operation wire being configured to perform a reciprocating movement in a longitudinal direction of the insertion section;
a wire pulling portion formed protruding from the forceps raising base in a direction crossing the longitudinal direction, the wire pulling portion being configured to hold a distal end of the operation wire and to transmit a pull force of the reciprocating movement of the operation wire to the forceps raising base; and
a distal end protrusion portion provided on the distal end structural portion, the distal end protrusion portion including a guide surface that guides the reciprocating movement of the operation wire, and a restriction portion provided at an end portion of the guide surface to restrict a rotation angle by which the wire pulling portion is able to turn with the forceps raising base.

Example 2. The endoscope according to Example 1, wherein the distal end protrusion portion extends more to a distal end side in the longitudinal direction than the second opening portion.

Example 3. The endoscope according to Example 2, wherein the second opening portion, the guide surface, the restriction portion, and the wire pulling portion are each provided on a straight line extending along the longitudinal direction.

Example 4. The endoscope according to Example 1, wherein the restriction portion restricts an angle of turning of the forceps raising base by abutting the wire pulling portion turning with the forceps raising base within a range of a predetermined rotation angle.

Example 5. The endoscope according to Example 4, wherein the restriction portion includes, at different positions, a first abutting portion that abuts a first part of the wire pulling portion when the forceps raising base is in a raised state, and a second abutting portion that abuts a second part of the wire pulling portion different from the first part when the forceps raising base is in a lowered state.

Example 6. The endoscope according to Example 1, wherein
the forceps raising base includes a contact surface that comes into contact with the treatment instrument and guides the treatment instrument, and
the wire pulling portion is provided at a position offset from the contact surface in a direction crossing the longitudinal direction.

Example 7. The endoscope according to Example 6, wherein the forceps raising base and the wire pulling portion are integrally formed.

Example 8. The endoscope according to Example 7, wherein the wire pulling portion includes a surface that is integrally joined to the contact surface of the forceps raising base.

Example 9. The endoscope according to Example 1, wherein the guide surface of the distal end protrusion portion is a curved surface.

Example 10. The endoscope according to Example 9, wherein the curved surface is formed into a shape that extends along a trajectory of the operation wire that is connected to the wire pulling portion after extending from the second opening portion when the forceps raising base is in a lowered state.

Example 11. The endoscope according to Example 9, wherein the curved surface is formed with a gap to the wire pulling portion to prevent the wire pulling portion from coming into contact with parts other than the restriction portion in a range where the wire pulling portion moves according to a turning operation of the forceps raising base.

Example 12. The endoscope according to Example 1, wherein
the distal end of the operation wire includes a large diameter portion with an increased diameter,
the wire pulling portion includes a wire engaging portion where the large diameter portion is removably engaged, and
the forceps raising base is raised by a pull force that is transmitted from the large diameter portion to the wire pulling portion via the wire engaging portion, when the operation wire is pulled in a state where the large diameter portion is engaged with the wire engaging portion.

Example 13. The endoscope according to Example 1, wherein the first opening portion and the second opening portion are each disposed at different positions across one surface of the distal end protrusion portion.

Example 14. The endoscope according to Example 1, wherein a surface, of the distal end protrusion portion, facing the forceps raising base is formed smoothly in a range of a turning operation of the forceps raising base.

Example 15. The endoscope according to Example 1, wherein parts, of the distal end protrusion portion and the forceps raising base, facing each other are disposed including a gap that is, even at a largest part, smaller than a dimension of the first opening portion.

Example 16. The endoscope according to Example 1, further comprising a distal end cover that is attached to the distal end structural portion to cover the forceps raising base, the operation wire, the wire pulling portion, and the distal end protrusion portion.

Example 17. The endoscope according to Example 1, wherein the endoscope is a single-use endoscope that is disposed of after being used once.

What is claimed is:

1. An endoscope, comprising:
an insertion section including a distal end portion, a bending portion, and a flexible tube portion,
wherein the bending portion is between the distal end portion and the flexible tube portion,
wherein the flexible tube portion is at a proximal end side of the insertion section and the distal end portion at a distal end side of the insertion section;
wherein the distal end portion includes:
a first opening connected to an instrument channel,
a second opening connected to a wire channel,
a wire located in the wire channel and protruding distally from the second opening,
a first detent surface, and
a raising base rotatable about an axis between a lowered position and a raised position,
wherein, in the raised position, the raising base intersects a projection of an axis of the instrument channel extending from the first opening,
wherein the raising base includes an attachment housing and a distal end of the wire is attached to the raising base at the attachment housing,
wherein the wire is movable within the wire channel to rotate the raising base about the axis from the lowered position to the raised position, and
wherein, in the raised position, a stop surface of the attachment housing contacts the first detent surface to limit an amount of rotation of the raising base.

2. The endoscope according to claim 1, wherein the distal end portion further includes a guide surface,
wherein the guide surface is located distally relative to the first detent surface, and
wherein a portion of the attachment housing travels over the guide surface during rotation of the raising base.

3. The endoscope according to claim 2, wherein the guide surface extends from the first detent surface in a direction of a longitudinal axis of the insertion section, and
wherein the guide surface includes a curved surface portion.

4. The endoscope according to claim 3, wherein the curved surface portion curves downward relative to the longitudinal axis of the insertion section.

5. The endoscope according to claim 3, wherein the guide surface includes a proximal end surface portion, and
wherein the first detent surface and the proximal end surface portion intersects at a right angle or at an acute angle.

6. The endoscope according to claim 2, wherein the guide surface is on a guide body extending distally relative to the second opening, and
wherein the guide body further includes a first side surface oriented toward a space between the raising base and the first opening.

7. The endoscope according to claim 6, wherein rotation of the raising base occurs along a first rotation path,
wherein the first opening has a first diameter,
wherein the first side surface of the guide body is separated from the first rotation path by a gap, and
wherein a length of the gap is smaller than a length of the first diameter.

8. The endoscope according to claim 1, wherein the distal end portion has a central axis extending longitudinally from a proximal end to a distal end, and
wherein, in a cross-section perpendicular to the central axis, each of the second opening, the first detent surface, and the attachment housing are located closer to a periphery of the cross-section than the first opening is located.

9. The endoscope according to claim 1, wherein the distal end portion has a central axis extending longitudinally from a proximal end to a distal end,
wherein, in a cross-section perpendicular to the central axis: (i) the first detent surface is at a first distance from the axis of the instrument channel, (ii) the second opening is at a second distance from the axis of the instrument channel, and (iii) a radius of the instrument channel has a radial length, and
wherein the first distance, and the second distance are each larger than the radial length.

10. The endoscope according to claim 1, wherein the first detent surface restricts a movement of the raising base in a proximal direction.

11. The endoscope according to claim 1, wherein the distal end portion further includes a second detent surface, and
wherein, in the lowered position, the attachment housing contacts the second detent surface.

12. The endoscope according to claim 11, wherein the first detent surface restricts movement of the raising base in a first rotation direction,
wherein the second detent surface restricts movement of the raising base in a second rotation direction, and
wherein the first rotation direction is opposite to the second rotation direction.

13. The endoscope according to claim 12, wherein the distal end portion further includes a guide surface,
wherein the guide surface extends between the first detent surface and the second detent surface, and
wherein a portion of the attachment housing travels over the guide surface during rotation of the raising base.

14. The endoscope according to claim 1, wherein the raising base further includes a deflecting surface and a side surface,
wherein, in the raised position, the deflecting surface intersects the projection of the axis of the instrument channel extending from the first opening, and
wherein the side surface includes the attachment housing.

15. The endoscope according to claim 1, wherein the attachment housing is integrated into a body of the raising base.

16. The endoscope according to claim 1, wherein the attachment housing includes a seating fixture,
wherein the distal end of the wire includes a termination, and
wherein the termination engages with the seating fixture to attach the distal end of the wire to the raising base.

17. The endoscope according to claim 1, wherein the distal end of the wire is detachably attached to the raising base at the attachment housing.

18. The endoscope according to claim 1, further comprising a distal end cover attached to the distal end portion.

19. The endoscope according to claim 1, wherein the endoscope is a single-use endoscope.

20. The endoscope according to claim 1, wherein the first detent surface includes a flat surface portion, wherein the flat surface portion is normal to a longitudinal direction of the insertion section.
